(19) United States
(12) Patent Application Publication
(10) Pub. No.: US 2008/0306315 A1
(43) Pub. Date: Dec. 11, 2008

(54) Metal-Organic Framework Catalysts and Their Use in Hydrocarbon Transformation

(75) Inventors: Karl-Petter Lillerud, Oslo (NO); Mats Tilset, Oslo (NO); Unni Olsbye, Oslo (NO); Kai Chung Szeto, Oslo (NO); Morten Bjorgen, Oslo (NO); Kjell Ove Kongshaug, Oslo (NO); Silvia Bordiga, Oslo (NO); Jasmina Hafizovic, Oslo (NO); Alexander Krivokapic, Oslo (NO); Soren Jakobsen, Oslo (NO)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITETET I OSLO, Oslo (NO)

(21) Appl. No.: 11/995,466

(22) PCT Filed: Jul. 14, 2006
(86) PCT No.: PCT/GB2006/002623

§ 371 Date: Aug. 14, 2008

(30) Foreign Application Priority Data
Jul. 14, 2005 (GB) 0514478.7

(51) Int. Cl. C07C 5/00; B01J 31/12

(52) U.S. Cl. 585/250; 502/150

(57) ABSTRACT

The invention relates to a porous heterogeneous catalyst material comprising frameworks of inorganic cornerstones connected by organic bridges, characterized in that as organic bridges are used ligands having a complexed catalytically active metal. The metals are preferably palladium and platinum. The ligands preferably contain nitrogen donor groups for complexing the catalytically active metal and carboxylate groups connecting to the inorganic cornerstones.

This invention relates to novel heterogeneous catalysts, their preparation, compositions containing them, and their use.

Traditionally, catalysts are considered to fall into two groups, the homogeneous and the heterogeneous catalysts. Homogeneous catalysts are ones which are in the same phase as the reactants, while heterogeneous catalysts are ones which are in a different phase, e.g. a solid phase when the reactants are in a liquid or gas phase.

In industrial applications, heterogeneous catalysts are often preferred over homogeneous catalysts and accordingly many homogeneous catalysts are heterogenized by being loaded onto solid supports, e.g. porous silica as is conventionally done in the olefin polymerization industry. One reason for the preference for heterogeneous catalysts over homogeneous catalysts is that product/catalyst separation is easier. Another is that, for gas phase reactions, it is often simpler to carry out a continuous reaction by flowing the reactant gas phase over a static solid catalyst or through a fluidized bed of catalyst particles.

Nonetheless, heterogeneous catalysts do have their shortcomings, for example they may be chemically ill-defined and may thus present a plurality of different types of catalytically active sites, each with a different reactivity or selectivity for the reaction system. These characteristics make it difficult to rationally improve such catalysts.

Homogeneous catalysts are often organometallic complexes which may be synthesized by procedures which allow production of compounds with highly uniform active sites, and the activity and selectivity of such sites may be adjusted by selective modification of the organic and/or metallic components of the complex. The predictable “single-site” nature of such homogeneous catalysts is their main advantage over heterogeneous catalysts and thus there is an ongoing demand for procedures for the heterogenization of homogeneous catalysts without loss of their single site nature.

Probably the most advanced technique for heterogenization of homogeneous catalysts currently in use is the so-called “grafting” technique in which a catalytically active organometallic complex is adsorbed on a solid surface, most frequently a silica particle, especially a porous silica particle. This technique however has certain drawbacks, in particular: the technique relies on attachment to suitable surface sites on the support thus limiting the number of active sites per gram of catalyst; the surface of the support may not be homogeneous and the organometallic complex may attach in different ways at different surface sites leading to different activities or different specificities for the grafted complexes; grafting may involve displacement of one or more of the ligands of the organometallic complex which may result in a dual or multi-site rather than a single-site catalyst; and when grafting involves ligand displacement, the altered ligand pattern may result in a changed activity or selectivity relative to the heterogeneous catalyst.

These problems are described for example by Copéret et al., Angew. Chem. Int. Ed. 42: 156 (2003).

An alternative approach to heterogenization has been to build catalytically active metals into the framework of a porous three-dimensional material. Thus, for example, Mori et al ., in Microporous and Mesoporous Materials 73: 31 (2004), described the preparation of a microporous material having dinuclear Rh-O cornerstones held together by organic bridges. The rhodium sites in this material were shown to be active for catalysing alkene hydrogenation at low temperatures.

The approach adopted by Mori et al. however has little flexibility in that the metal centres at the cornerstones must function both as cornerstones (i.e. to hold the structure together) and as providers of the catalytically active site.

We have now found that this missing flexibility may be provided by incorporating the catalytically active metal into the organic bridges of such inorganic cornerstone/organic bridge structures. This may readily be done by providing the ligands on the basic homogeneous catalyst with extra functional groups capable of bonding to (e.g. complexing) the inorganic cornerstones.

Thus viewed from one aspect the invention provides a porous heterogeneous catalyst material comprising a framework, preferably a three-dimensional framework, of inorganic cornerstones connected by organic bridges, characterized in that as organic bridges are used compounds having a complexed catalytically active metal.

The materials of the invention may be stacked, chained structures or stacked, laminar, structures, i.e. like graphite; however they are more preferably structures in which the layers are held apart by the organic linker bridges, i.e. a porous, three dimensional, structure more akin to a zeolite than to the laminar structure of graphite.

For the avoidance of doubt, it should be pointed out that the term catalyst as used herein relates to materials which are catalytically active or which are catalyst precursors or cocatalysts, e.g. materials which become catalytically active on reaction with a catalyst activator. The use of cocatalysts and catalyst activators is well known in the field of catalysis. Catalyst activation, in the case of organometallic catalysts frequently involves loss of one or more ligands to expose one or more coordination sites on the metal. Thus for example the metallocene catalysts used in olefin polymerization are activated by reaction with an aluminoxane to remove the inorganic ligands. There are many other cases where a co-catalyst is used in catalyzed reactions. Thus, for example, in the Wacker process for oxidizing ethene to acetaldehyde using a Pd(II) (e.g. PdCl₂) catalyst, the catalyst is reduced to Pd and thus Cu(II) (e.g. CuCl₂) is used as a co-catalyst to reactivate the palladium to Pd(II), itself becoming reduced to Cu(I). Likewise in the solution phase oxidation of methane to methanol using a Pt(IV) (e.g. PtCl₄) catalyst, Cu(II) (e.g. CuCl₂) is also used as a co-catalyst to reoxidize the platinum. In other cases a co-catalyst may displace one of the ligands on the catalytic metal; thus for example HSO⁻₄ may be used to displace chloride ligands in the (bpym)PtCl₂ catalyst used for methane oxidation. This of course is comparable to the use of aluminoxanes mentioned above. It will be appreciated therefore that the complexed catalytically active metals in the organic bridges in the catalyst material of the invention may be in a pre-catalyst form which requires ligand removal or other activation for the material to enter its catalytically active form.

The invention is particularly attractive in that it provides a mechanism by which a compound, known to be active as a homogeneous catalyst, may be heterogenized by incorporation into a three-dimensional framework without loss of its active site(s).

The porosity of the material according to the invention that is required for it to be catalytically active may be achieved by solvent removal by drying at elevated temperature (i.e. ≧25° C., preferably 30 to 350° C., more preferably 35 to 250° C., especially 40 to 120° C.) and/or reduced pressure, e.g. at pressures below 1 bar, especially below 10 mbar, particularly below 0.1 mbar, more especially below 0.01 mbar, for example 0.0001 mbar.

Exposure to high vacuum (e.g. 0.0001 mbar) for about 5 to 30 minutes at ambient temperature generally is preferred.

Relative to drying at ambient temperatures and pressure this drying can result in a further weight loss of about 10% which is sufficient to achieve the porosity necessary for catalytic activity. The catalyst material of the invention before use thus preferably has a solvent content of no more than 5% wt., more preferably no more than 2% wt., particularly no more than 1% wt., especially no more than 0.5% wt. If only dried at ambient temperature and pressure, the catalyst material will generally have a residual solvent content of at least 10% wt. and will show little or no activity as a catalyst.

The complexed catalytically active metal in the materials of the invention is preferably a transition, lanthanide or actinide metal, most preferably a transition metal. Particular mention in this regard may be made of group 4 metals (e.g. Ti, Zr and Hf) which are especially useful for olefin polymerization, group 6 metals (e.g. Cr) which likewise are useful for olefin polymerization, group 8 metals (e.g. Fe, Ru and Os) which are useful for syngas production and olefin metathesis, group 10 metals (e.g. Ni, Pd and Pt) which are useful for hydrogenation/dehydrogenation, oxidation and hydroxylation reactions. The use of platinum, (particularly Pt(II)), ruthenium, osmium and palladium is especially preferred.

The bonding of the organic bridges to the inorganic cornerstones may be covalent or non-covalent, e.g. by complexation or by electron donation. Where the cornerstone is provided by a metal (such as gadolinium or yttrium for example), bonding is preferably by complexation, e.g. chelation. Where it is a pseudometal (such as silicon), bonding may preferably be covalent.

The inorganic cornerstone may be a single metal or pseudometal (e.g. Si) atom or a multi-atom moiety which does not include carbon and contains one or more metal or pseudometal atoms. Especially preferably the cornerstone contains one or more transition, lanthanide or actinide metal atoms, optionally together with one or more group 16 atoms (e.g. O and S). Group 13 and 14 metals, e.g. Al, Ge and Ga may also be used, as may group 2 metals, e.g. Ba. Complexable metal/non-metal clusters are well known, e.g. MₙAₘ clusters where M is a transition, lanthanide or actinide metal atom, A is selected from group 16 and/or 15 atoms and n and m are positive integers, for example vanadium oxides (e.g. VO), tungsten oxides, rhodium oxides (e.g. RhO), aluminophosphates (e.g. AlPOₓ) etc. Particularly preferably however the cornerstone is a transition, lanthanide or actinide metal in oxidation state III, e.g. Hf, Zr, Ti, Y, Sc, La, Gd, Sm, Dy, Ho and Er, especially Y. The use of transition metals in oxidation state II, e.g. Cu, is also preferred. Metals such as Zn, Cd, Ni, Co, Mn, Ba, Ce, and Nd may also be used. The use of Ce is especially preferred. If appropriate, the metal(s) of the inorganic cornerstone may be bonded to (e.g. complexed by) a moiety (e.g. a ligand) which does not form a skeletal part of the framework of the material, i.e. which does not form part of a bridge to another cornerstone. In this event the moiety may be carbon-containing or non-carbon containing.

Particularly desirably, the metal(s) or pseudometal(s) of the cornerstone are not catalytically active in the reaction the material is intended to catalyse, either by virtue of intrinsic inactivity or by virtue of their complexation or oxidation state. However, if desired, the metal(s) of the cornerstone may be less active than those in the bridges (e.g. >50% less active, preferably >90% less active) or they may also be active. The inclusion of catalytically active metals in the cornerstones is of particular interest for olefin polymerization catalysts as the monomodal molecular weight distribution produced using a single site catalyst is often significantly less preferred than the bimodal molecular weight distribution produced using a combination of single site catalysts. Thus for example it may be desirable to combine a group 4 metal in the bridges with a group 4 metal in the cornerstones.

In the materials of the invention, the organic bridges may wholly or only partially (e.g. ≧10%) be bridges containing catalytically active metals. While the activity per gram catalyst is increased as the proportion of bridges which are catalyst metal containing is increased, the inclusion of bridges which are not catalyst metal containing may enhance the stability of the three dimensional structure, especially where the bridge backbone includes the metal (ie. where the cornerstone to cornerstone linkage is via two ligands each binding to a cornerstone and the catalytically active metal). Where a catalyst metal free bridge is used, this may be any difunctional compound capable of binding to two cornerstones, e.g. as in the materials described by Mori (supra). Particularly preferably, the cornerstone-coordinating groups in such compounds are the same form of functional group and are different to the functional groups which coordinate the catalytic metal in the catalytic metal containing bridges.

The organic bridges must be at least bifunctional to achieve a bridging effect; however a higher degree of functionality, i.e. so that one bridge may link more than two cornerstones, may be used if desired; for example the bridges may be tetrafunctional. In general however they should preferably be rigid so that collapse of the resulting framework is hindered. Nonetheless, the organic bridges are preferably linear bifunctional compounds, i.e. of formula I

Rᵢ-A-(Z)ₚ-R₁ (I)

where p is zero or 1; Z is a group of formula

—(R₂-M(L)ₘ-R2-A)-ₙ

or, more preferably,

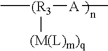

each R₁ is a binding (e.g. coordinating) group or a precursor therefor; A is a bond or a linear backbone, optionally including fused rings and/or pendant side chains; n is zero or a positive integer (generally 1, 2 or 3, preferably 1); M is the catalytically active metal; m is zero or a positive integer the value of which is determined by the identity, oxidation state and coordination geometry of M; each L, which may be the same or different, is a group coordinating or dissociated from M which group may also be attached to an A moiety; R₃ is a group coordinating one or more M(L)ₘ groups; q is a positive integer (e.g. 1, 2 or 3, preferably 1 or 2); and each R₂ is an M-coordinating group; where R₁ and R₂ or R₃ are preferably different.

The coordinating groups in the organic bridges (i.e. groups R₁ and R₂/R₃ in formula I above) may respectively be any groups capable of forming bonds to the inorganic cornerstones that are sufficiently strong for the catalytic material to have structural integrity during its use as a catalyst and any groups capable of presenting the catalytic metal in a catalytically active conformation, preferably as a “single-site” conformation. In general, the cornerstone coordinating groups (CCGs) will be mono- or poly-dentate (e.g. bi-dentate) and will complex via an oxygen, nitrogen or sulfur atom, particularly preferably an oxygen atom or a pair of oxygen atoms (e.g. where R₁ is a carboxyl or other oxyacid group). Mono- or bi-dentate groups are preferred as in order to achieve a three-dimensional framework the inorganic cornerstones must be coordinatable by at least three and preferably at least 4 (e.g. 4, 5, 6, 7 or 8) CCGs. The catalytic metal coordinating groups (CMCGs) may be mono- or poly-dentate and typically may complex via σ, π or η bonds. Typically the CMCGs will contain atoms from group 15 (e.g. N, P, etc.), e.g. they maybe nitrogen-containing components of a moiety with a delocalized electron structure, or oxyacid groups (or the sulphur analogs) such as carbon or phosphorus oxyacids, or η bonding groups (such as cyclopentadienyl or indenyl groups), although where the complexation of the metal occurs at multiple positions further CCMGs such as amino, hydroxyl or thiol groups, may be present. Preferably the bond strength between the CCMGs and the metal will be stronger than would be the bond strength between the CCGs and the metal unless the framework is constructed before the catalytic metal is loaded on. In an especially preferred embodiment of the invention, the CCMGs are nitrogen-based and the CCGs are oxygen-based (e.g. oxyacid groups such as carboxyl groups). Preferred such linear organic bridge molecules, suitable in particular where the catalytic metal is Pt(II), are described for example by Lehn et al., in JOC 62: 5458 (1997) in particular in FIGS. 17 and 18 therein. The contents of Lehn (supra) are hereby incorporated by reference.

The backbone of the organic bridge molecules, and the chemical nature of the cornerstones, may both be selected so as to achieve the desired spacing between active sites in the material and indeed so as to confer rigidity to the material. Typically, the organic bridges, when in place, will provide a bridge 5 to 50, more preferably 8 to 30, especially 12 to 20, atoms long between linked inorganic cornerstones. (Bridge length in this context means the shortest countable and may include the catalytic metal, in particular where the bridge is formed by two organic bridge molecules each coordinated to a catalytic metal atom). With the exception of the catalytic metal, the backbone atoms will generally be selected from C, N, O, S, P and Si, typically at least two being N, O or S and at least 3 being C. Bridge rigidity may be enhanced by incorporation of cyclic groups, in particular unsaturated 5 to 7 membered rings, and/or by substitution. To confer enhanced rigidity on the framework, the cornerstone preferably involves a single metal (or pseudo metal) bonded to the organic bridges with that metal or pseudometal being selected from row 5 or earlier in the periodic table.

Rigidity of the organic bridge may be achieved by selecting a compound in which rotation about the bonds between the CCGs is denied by π-bonding, or is sterically inhibited, or does not affect the spacing between the CCGs. Particularly preferably the backbone components between the CCGs are made up of cyclic or acyclic groups, especially such groups including groups having two nitrogens separated by two carbons and thus available to to coordinate the catalyst metal. Examples of such groups include 2,2′-bipyridines, N,N′-bisphenyl-ethylenediimines, 2,2′-bispyrimidines, 3,6-bis(pyridin-2-yl)-pyridazines, 1,10-phenanthrolines and 13,14-diazapentaphenes. Such groups advantageously have CCGs (e.g. carboxyl groups) at their ends. Thus typical organic compounds useful in this regard include 1,10-phenanthroline-3,8 dicarboxylic acid; 3,8-bis(4-carboxyphenyl) 1,10 phenanthroline; 13,14 diazapentaphene -3,10-dicarboxylic acid; 6-(5-carboxypyridin-2-yl) nicotinic acid (BPDC); 1,4-bis(4-carboxyphenyl)-2,3-dimethyl-1,4-diazabutadiene; 2,2′-bipyrimidine -5,5′-dicarboxylic acid; 2-(5-carboxypyrimidin-2-yl)pyrimidine-5-carboxylic acid; and 6-(6-(5-carboxypyridin-2-yl)pyridazin-3-yl) nicotinic acid.

While the use of BPDC is exemplified herein, other organic such ligands may be used to advantage. BPDC is available commercially, e.g. from Aldrich.

Thus a preferred group of organic ligands comprises compounds with two carboxyl groups (as CCGs) and two nitrogens separated by two carbons and in a delocalized electron system (as CCMGs). Where more than one metal is to be bound, the ligand may contain more than one such NCCN group. The NCCN groups preferably occur within a fused ring system or on two adjacent but non-fused rings. They may however occur in a non-cyclic system; however in this event they are preferably attached to one or more aromatic rings.

The organic bridge molecules are preferably analogs of ligands present as homogeneous organometallic catalytic compounds, analogs that is in the sense that the molecule is modified to include a group capable of coordinating the inorganic cornerstones. Such a CCG is preferably placed on the molecule in such a way as to have minimal impact on the coordination geometry of the catalytic metal, especially in its activated state. Thus for example where the homogeneous catalyst is a bridged bis indenyl metallocene, the CCG or CCGs may be placed on groups pendant from the bridge or on groups pendant from the C₆ rings.

Examples of organic bridge molecules analogous to ligands in known homogeneous catalysts are set out in Table 1 below:

TABLE 1

| Homogeneous catalyst ligand type | Substitute ligand for heterogeneous analogs |
|---|---|
| 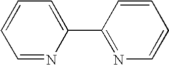 | 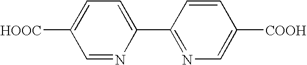 |
| 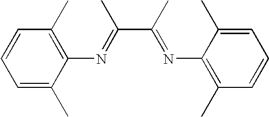 | 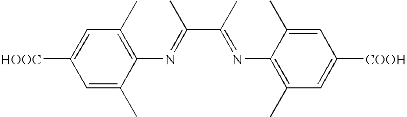 |
| 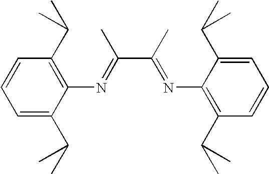 | 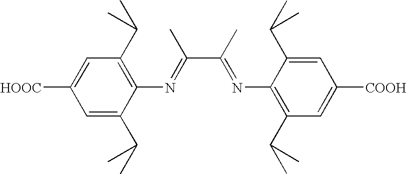 |
| 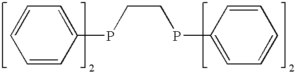 | 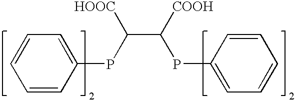 |
| 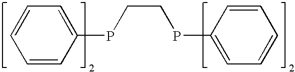 | 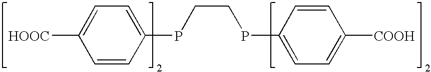 |
| 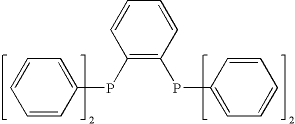 | 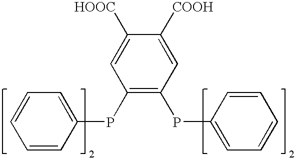 |
| 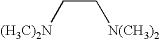 | 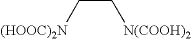 |
| 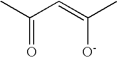 | 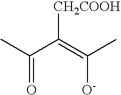 |
| 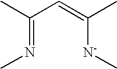 | 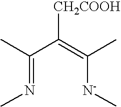 |
| 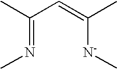 | 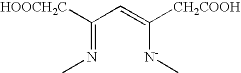 |
| 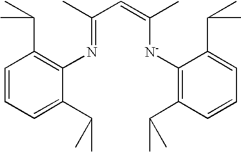 | 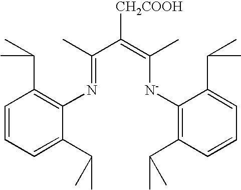 |
| 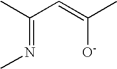 | 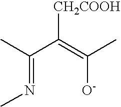 |

The catalytic material of the invention may be prepared by constructing the framework and then loading it with the catalytic metal (generally by a transmetallation reaction) or by reacting a complex of the catalytic metal with the inorganic cornerstone, or a precursor thereof, typically in a solvent or solvent mixture (e.g. water or an organic solvent such as an alcohol or ketone etc.), followed by solvent stripping, typically under reduced pressure. Such processes form a further aspect of the invention. Thus viewed from this further aspect the invention provides a process for preparing a catalyst material according to the invention, said process comprising complexing an inorganic cornerstone with a multidentate organometallic ligand (i.e. a ligand capable of binding at least two cornerstones and preferably a bidentate ligand) in a liquid solvent, preferably at elevated temperature and ambient or reduced pressure (e.g. 25 to 250° C., and 10 mbar to 20 bar, for example 25 to 110° C., and 10 mbar to 1 bar), optionally transmetallating the product to introduce a catalytically active metal, and removing the solvent.

Particularly preferably the organometallic ligand used is itself a homogeneous catalyst.

Where the catalyst material is prepared by complexing the cornerstones with an organometallic ligand, this ligand can itself be prepared by metallation of an organic ligand, optionally with the groups intended to complex the cornerstones in a protected or deactivated form so as to prevent them from coordinating to the metal. Such partial protection of complexing groups is achieved more readily when the CCGs and CCMGs are of chemically different types, e.g. amines and carboxyls. Such protection and subsequent deprotection before reaction to form the material's framework may be carried out using conventional chemical techniques.

The organic ligand itself may be constructed using conventional organic chemistry techniques or in certain cases may be available commercially.

The groups not forming part of the material's basic framework structure, e.g. groups which are displaced from the catalytic metal on activation or which otherwise “fill” empty coordination sites on the cornerstones may be any convenient groups, (for example halides, nitrates, and organic cations or optionally ionized solvent species, e.g. H₂0, DMF, EMF, alcohols, TMA⁺, TEA⁺) and may be present in the reagents used in the framework generating reaction or may be subsequently introduced, e.g. by ion or solvent exchange. Such groups may thus be introduced after framework construction and optionally before solvent removal, for example by “washing”, e.g. with a solvent which is more volatile than the one used for the framework construction reaction.

Thus the overall structure of the catalytic material may be seen to be of cornerstones (IC) connected to each other to form a three-dimensional framework by metallated or unmetallated bridges, typically exemplified by the simplified formulae II, III, and, less preferably, IV.

—R₁-A-R₃-A-R₁-(M)ₙ II

—R₁-A-R₂-M-R₂-A-R₁— III

—R₁-A-R₁— IV

where R₁ are CCGs, R₂ are CCMGs, A is a carbon containing backbone, M is a metal (either the catalytic metal or a metal displaceable by the catalytic metal), and n is zero or a positive integer (e.g. 1 or 2).

In a particularly preferred embodiment, the catalyst materials of the invention are heated, e.g. by microwave irradiation or in an oven, following the reaction to create the three-dimensional framework. This is particularly beneficial when the catalytic metal in the material is platinum.

Following solvent removal, the catalyst material may be further treated, e.g. pelletized, activated, prepolymerized, or formulated together with other materials, e.g. catalyst activators, binders, etc. Compositions comprising the catalyst material of the invention together with such other materials form a further aspect of the invention.

The pore size in the catalyst material may be varied according to need, e.g. in order to allow ready penetration of the reactants of the reaction it is to serve as a catalyst for. Thus for example platinum-based catalysts according to the invention are particularly suitable for use in hydrocarbon transformation (e.g. dehydrogenation or hydroxylation) and the pore size may be adapted to suit the size of the hydrocarbon starting material. Methods for the production of micro-and nanoporous materials, e.g. in the forms of granules or micro-or nano-particles are known in the art and may be used in this respect. See for example Dautzenberg, Catalyst Reviews—Science and Engineering 46: 335-338 (2004); Glaeser et al. “The application of zeolites in catalysis” in Springer Series in Chemical Physics No. 75, pages 161-212 (2004); Noack et al., Handbook of Porous Solids 4: 2433-2507 (2002); Maesen et al, “The zeolite scene—an overview” in Studies in Surface Science and Catalysis No. 137 (2nd Edition, 2001) pages 1 to 9; and Dabrowski et al., NATO Science Series IV, Earth and Environmental Sciences No. 24, pages 225-298 (2003); all of which are hereby incorporated by reference.

Preferred substrates for the catalyst materials of the invention, i.e. reagents for the reactions they catalyse, include hydrocarbons and hydrocarbon mixtures, e.g. natural gas, oil, aliphatic hydrocarbons, aromatic hydrocarbons, etc. Particularly preferred substrates include methane, ethane, propane, butane and isobutane and alkenes, e.g. C₂₋₆ alkenes.

Thus viewed from a further aspect the invention provides the use of a catalyst material according to the invention as a catalyst, particularly for hydrocarbon transformation (e.g. hydrogenation, dehydrogenation or hydroxylation or alkene activation or functionalization).

Viewed from an alternative aspect the invention provides a process for the catalysed transformation of a hydrocarbon (e.g. hydrogenation, dehydrogenation or hydroxylation or alkene activation or functionalization), characterized in that as a catalyst therefor is used a catalyst material according to the invention, optionally following catalyst activation.

Beside being useful as catalysts, the novel structure of the materials of the invention is suitable for presentation of metal atoms to a surrounding fluid for different purposes, e.g. ion exchange or as MR contrast agents (where the metal would be in a paramagnetic state, such as Gd(III) or Dy(III)) or as sensors (e.g. to show a colour change on exposure to particular chemical environments), and as adsorbents. Such other materials and their use form further aspects of the present invention.

In an analogous fashion, organic cornerstones (e.g. C₂ moieties) may be used in place of inorganic cornerstones to link the catalytic-metal containing organic bridges together into a three-dimensional framework. In this case, the organic bridge to organic cornerstone bonding is preferably covalent. The bridge to cornerstone bonding may readily be achieved using electrophilic or nucleophilic substitution reactions, e.g. using unsaturated or halogen- (or other leaving group-) substituted cornerstone precursors. Such organic cornerstoned materials and their uses form further aspects of the present invention.

The present invention will now be described further by the following non-limiting Examples and with reference to the accompanying drawings, in which:

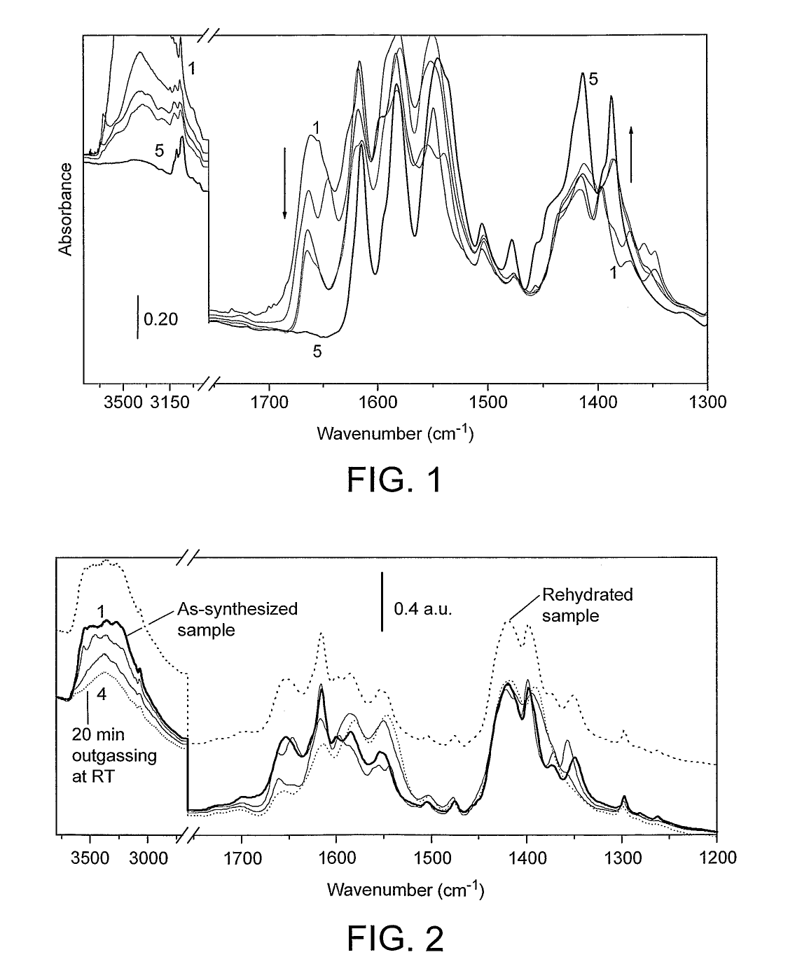

FIGS. 1 and 2 are Fourier transform infrared spectra of the compounds of Examples 1 and 6 showing the change in spectrum as solvent removal proceeds from 1 (as synthesized) to 4 (after 20 minutes exposure to high vacuum);

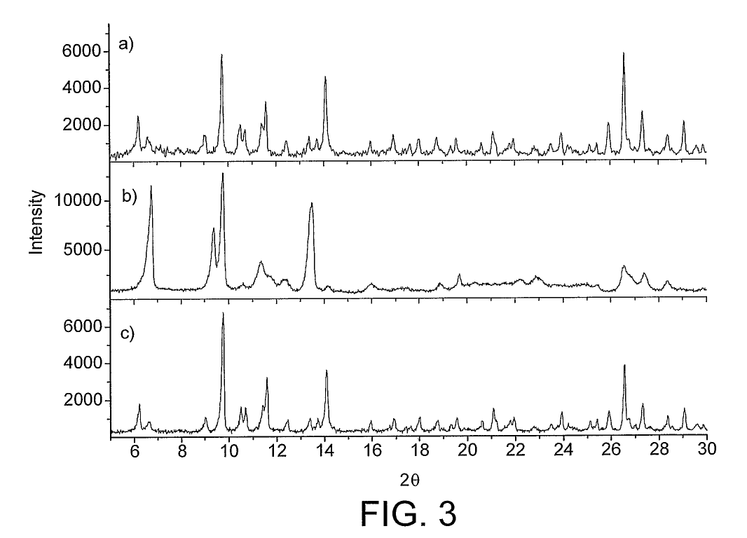

FIG. 3 shows powder X-ray diffraction patterns for the compound of Example 6 before (a) and after (b) solvent removal and (c) after solvent replacement;

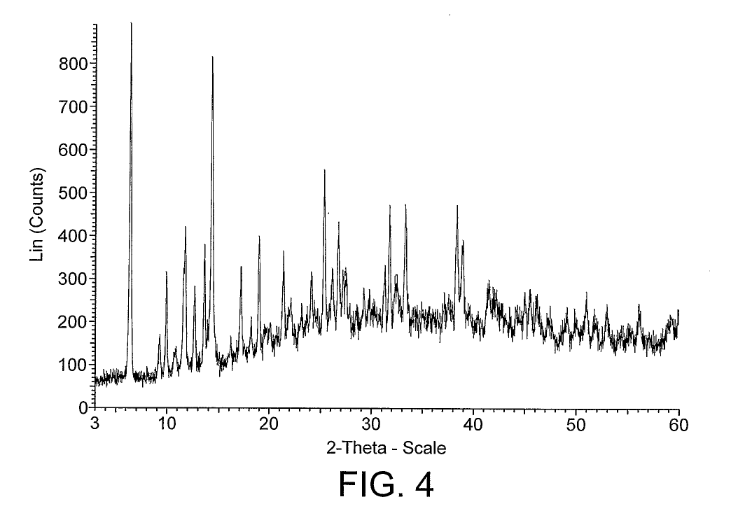

FIGS. 4, 5 and 6 show the powder X-ray diffraction patterns for the products of Examples 4, 6 and 18; and

FIG. 7 is a plot of weight loss against temperature for the material of Example 6; and

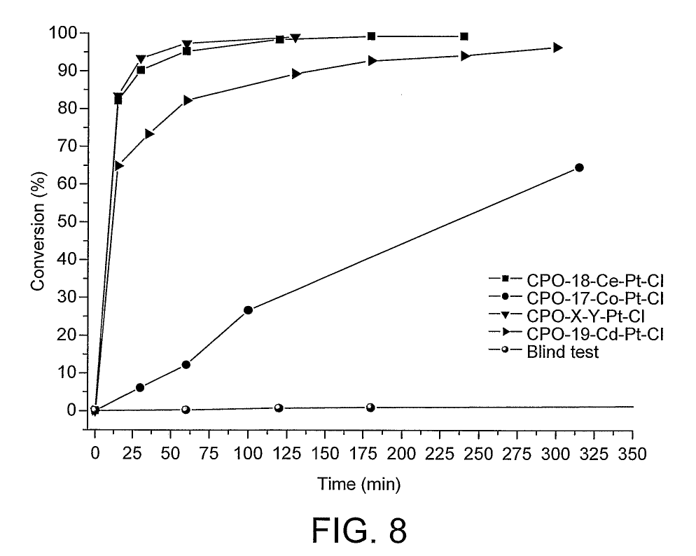

FIG. 8 is a plot of ethene to ethane conversion over time using four catalysts according to the invention and using no catalyst (control).

In the following Examples, the number following CPO is an arbitrary designation and does not refer to the stoichiometry of the compounds in question.

### Example 1

### Synthesis of CPO-11-Y—Pt—Cl

0.12 g K₂PtCl₄, 0.06 g BPDC, 0.37 g Y(NO₃)₃.6H₂O and 8.50 g distilled water were mixed in a Teflon-lined vessel. The vessel was placed in a sealed autoclave and heated to 100° C. (heating rate: 80° C./min) for 10 hours with continuous stirring. The title product was isolated after quenching to room temperature, filtration, washing with distilled water and drying in air at ambient temperature (yield: 76.4% based on Pt).

### Example 2

### Synthesis of CPO-11-Y—Pt—Cl

0.08 g K₂PtCl₄, 0.05 g BPDC, 0.28 g Y(NO₃)₃6H₂O and 6.59 g distilled water were mixed in a Teflon-lined vessel. The vessel was placed in a sealed stainless steel autoclave. The autoclave was left for 13 hours in a commercial oven set at 100° C. The product was isolated after quenching to room temperature, filtration, washing with distilled water and drying in air at ambient temperature.

### Example 3

### Synthesis of CPO-11-Y—Pt—Cl

0.10 g K₂PtCl₄, 0.06 g BPDC, 0.06 g Y(NO₃)₃.6H₂O and 8.78 g distilled water were mixed in a Teflon-lined vessel. The vessel was placed in a sealed autoclave and heated to 100° C. (heating rate: 80° C./min) for 10 hours with continuous stirring. The product was isolated after quenching to room temperature, filtration, washing with distilled water and drying in air at ambient temperature.

### Example 4

### Synthesis of CPO-11-Y—Pt—Cl

0.09 g K₂PtCl₄, 0.05 g BPDC, 0.32 g Y(NO₃)₃.6H₂O and 9.56 g distilled water were mixed in a Teflon-lined vessel. The vessel was placed in a sealed autoclave and heated to 100° C. (heating rate: 80° C./min) for 12 hours with continuous stirring. The product was isolated after quenching to room temperature, filtration, washing with distilled water and drying in air at ambient temperature (yield: 94.6%). The powder X-ray diffraction pattern of this is shown in FIG. 4.

### Example 5

### Synthesis of CPO-11-Y—Pt—Cl

0.10 g K₂PtCl₄, 0.06 g BPDC, 0.10 g Y(NO₃)₃.6H₂O and 8.77 g distilled water were mixed in a Teflon-lined vessel. The vessel was placed in a sealed autoclave and heated to 100° C. (heating rate: 53° C./min) for 9 hours with continuous stirring. The product was isolated after quenching to room temperature, filtration, washing with distilled water and drying in air at ambient temperature.

### Example 6

### Synthesis of CPO-11-Gd—Pt—Cl

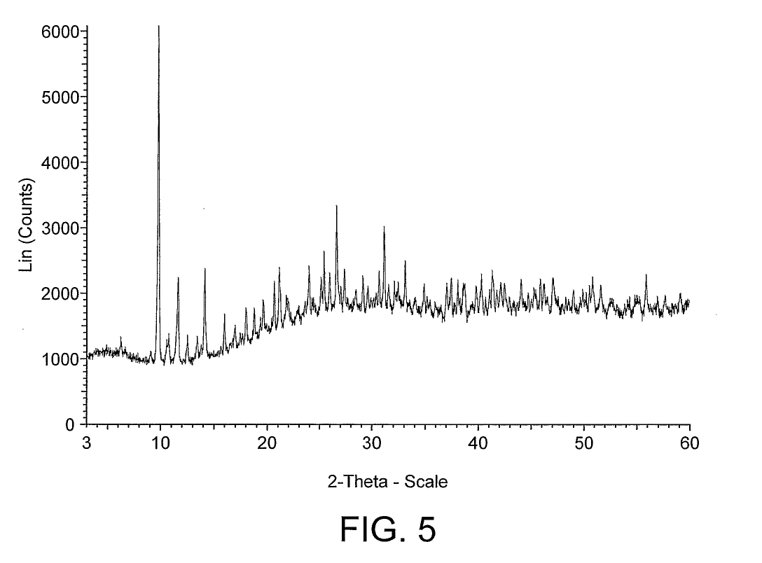

0.12 g K₂PtCl₄, 0.07 g BPDC, 0.10 g Gd(NO₃)₃.6H₂O and 8.73 g distilled water were mixed in a Teflon-lined vessel. The vessel was placed in a sealed autoclave and heated to 100° C. (heating rate: 40° C./min) for 9 hours with continuous stirring. The product was isolated after quenching to room temperature, filtration and washing with distilled water and drying in air at ambient temperature (yield: 86.6% based on Pt). The powder X-ray diffraction pattern of the product is shown in FIG. 5.

### Example 7

### Synthesis of CPO-11-Gd—Pt—Cl

0.11 g K₂PtCl₄, 0.07 g BPDC, 0.08 g Gd(NO₃)₃.6H₂O and 9.75 g distilled water were mixed in a Teflon-lined vessel. The vessel was placed in a sealed stainless steel autoclave. The autoclave was left for 16 hours in a commercial oven set at 105° C. The product was isolated after quenching to room temperature, filtration, washing with distilled water and drying in air at ambient temperature.

### Example 8

### Synthesis of CPO-11-Gd—Pt—Cl

0.12 g K₂PtCl₄, 0.07 g BPDC, 0.08 g Gd(NO₃)₃.6H₂O and 9.75 g distilled water were mixed in a Teflon-lined vessel. The vessel was placed in a sealed stainless steel autoclave. The autoclave was left for 16 hours in a commercial oven set at 101° C. The product was isolated after quenching to room temperature, filtration, washing with distilled water and drying in air at ambient temperature.

### Example 9

### Synthesis of CPO-11-Gd—Pt—Cl

0.10 g K₂PtCl₄, 0.06 g BPDC, 0.08 g Gd(NO₃)₃.6H₂O and 8.79 g distilled water were mixed in a Teflon-lined vessel. The vessel was placed in a sealed autoclave and heated to 100° C. (heating rate: 53° C./min) for 10 hours with continuous stirring. The product was isolated after quenching to room temperature, filtration, washing with distilled water and drying in air at ambient temperature.

### Example 10

### Synthesis of CPO-11-Gd—Pt—Cl

0.13 g K₂PtCl₄, 0.07 g BPDC, 0.09 g Gd(NO₃)₃.6H₂O and 8.72 g distilled water were mixed in a Teflon-lined vessel. The vessel was placed in a sealed autoclave and heated to 100° C. (heating rate: 26° C./min) for 6 hours with continuous stirring. The product was isolated after quenching to room temperature, filtration, washing with distilled water and drying in air at ambient temperature.

### Example 11

### Thermal Stability of the CPO-11-Y—Pt—Cl. Thermogravimetric Analysis

About 10 mg of the product of Example 1 was heated to 700° C. at 2° C./min in a flow of oxygen or nitrogen (12 mL/min). The thus obtained weight loss profiles showed a continuous weight loss (about 10%) in the temperature interval 25-110° C., representing removal of the solvent water. When the sample was heated in O₂, the subsequent weight loss, representing structural decomposition, started at about 380° C. In N₂, the final weight loss took place from about 420° C.

### Example 12

### Thermal Stability of CPO-11-Gd—Pt—Cl. Thermogravimetric Analysis

About 10 mg of the product of Example 6 was heated to 900° C. at 2° C./min in a flow of oxygen or nitrogen (12 mL/min). The weight loss profiles showed a continuous weight loss starting at room temperature and ending at 120° C. resulting from solvent removal. In this case, water loss resulted in a 9% weight reduction. The structural decomposition took place from about 320° C., regardless of the carrier gas, and clearly proceeded via several steps.

### Example 13

### Synthesis of CPO-12-Cu—Pt—Cl

0.07 g K₂PtCl₄, 0.03 g BPDC, 0.08 g CuSO₄.5H₂O and 29.81 g of distilled water were mixed in a Teflon-lined vessel. The vessel was placed in a sealed autoclave and heated to 100° C. (heating rate: 53° C./min) for 12 hours without stirring. The product was isolated after cooling to room temperature, filtration, washing with distilled water and drying in air at ambient temperature.

### Example 14

### Synthesis of CPO-12-Co—Pt—Cl

0.04 g K₂PtCl₄, 0.03 g BPDC, 0.12 g Co(NO₃)₂.6H₂O and 10 ml DMF were mixed in a Teflon-lined vessel. The vessel was placed in a sealed stainless steel autoclave. The autoclave was left for 12 hours in a fan-assisted commercial oven set at 100° C. The product was isolated after cooling in air to room temperature, filtration and drying in air at ambient temperature.

### Example 15

### The Removal of Solvent Water from CPO-11-Y—Pt—Cl at Room Temperature using Applied High Vacuum (0.0001 mbar)

A series of FTIR spectra were collected for the product of Example 1 at different stages during water removal and are shown in FIG. 1. The series was obtained by degassing the sample at room temperature for 30 s, 1 min, 5 min, and 20 min. The IR spectrum of the as-synthesized sample showed intense and unresolved absorptions extending from 3670 cm⁻¹ to 3000 cm⁻¹ due to ν(O—H) stretching modes of water. An intense band centered at 1660 cm⁻¹ represents the corresponding δ(OH) mode of adsorbed water. The stretching and bending modes were red- and blue-shifted respectively, indicating that water is engaged in medium strong hydrogen bonds. Upon degassing, a fast decrease in intensity of the band representing water stretching modes was observed. For intermediate pumping, water molecules with nearly free ν(O—H) bonds could also be seen at 3650 cm⁻¹. The final spectrum demonstrated a nearly total absence of water. Water can thus be completely removed either by heating (confirmed with TGA) or evacuation at high vacuum at room temperature.

### Example 16

### The Reversible Removal of Solvent Water from CPO-11-Gd—Pt—Cl at Room Temperature using Applied High Vacuum (0.0001 mbar)

FIG. 3 displays capillary powder X-ray diffraction patterns of: (a) the starting Pt—Gd material (i.e. the material of Example 6); (b) the dehydrated phase obtained by evacuation at room temperature (the sample was kept in this dehydrated form by sealing the capillary); (c) after exposing the previously evacuated sample to air. Exposing the dehydrated sample (diffraction pattern b) to air (diffraction pattern c) gives a diffraction pattern virtually indistinguishable from that of the starting material (diffraction pattern a). Clearly, upon reintroducing water to the dehydrated sample, an X-ray pattern with peak positions and intensities indistinguishable from those of the original solid is obtained, serving as evidence to the reversibility of the inclusion process. The material does not suffer any permanent structural changes after water desorption and re-adsorption. A series of FTIR spectra (see FIG. 2) for the product of Example 6 were obtained analogously to those discussed in Example 15 above. Spectrum 1 in FIG. 2 presents the vibrational properties of a wafer of the as-synthesized sample and spectra 2-4 report the successive degassing (from 10 s to 20 min) at room temperature. After spectrum 4 was collected, the sample was exposed to water, and the spectrum of the re-hydrated sample is represented by the upper dotted curve. Upon water removal (spectra 2-4), rather evident changes in the vibrational properties of the system are observed. The intensities of the water stretching and—bending modes are reduced (absorptions at 3700-2800 cm⁻¹ and around 1650 cm⁻¹, respectively). In addition, water removal changes both the intensities and positions of the framework vibrations (absorptions in the range 1640-1250 cm⁻¹), confirming that water molecules interacting with the framework indeed are removed. When the previously evacuated sample was exposed to water, the vibrational properties of the material were fully regained as shown by the upper dotted curve in FIG. 2. The FTIR data demonstrate a reversible water removal at room temperature.

Removal of water from the as-synthesized material gives a color change from bright red to dark brown/red, suggesting interactions between water and the metal centres in the framework. DRUV/VIS spectra were obtained of the as-synthesized sample; the dehydrated phase, and after reintroducing water. Metal-to-ligand charge transfers (MLCT) diagnostic for the Pt centre gave absorptions starting at 14000 cm⁻¹ for the as-synthesized sample. A rather evident change in the electronic properties of the material could be seen in this region when water was removed. The anhydrous phase gave an extended absorption in the visible part of the spectrum. Exposing the sample to water gave a spectrum fully coincident to that of the original sample, suggesting the water molecules re-occupied their initial positions.

The temperature and weight-loss profiles of a sample that was dehydrated by heating to 180° C. and subsequently exposed to water at room temperature was also recorded. Water removal resulted in a weight-loss of about 8.2%. After water re-adsorption, the mass of the sample was regained by 99.6%. This gives a clear indication that the sample can be fully dehydrated without suffering any loss in porosity.

### Example 17

### Interaction of Acetonitrile with Dehydrated Sample

6.7 hPa of CD₃CN was introduced into a cell containing a wafer of the dehydrated sample of the product of Example 1 and a series of spectra was subsequently collected. After a contact time of 80 minutes, the growth of the absorptions at 2290 cm⁻¹ and 2263 cm⁻¹ was observed. These observations demonstrate that the dehydrated material interacts significantly with acetonitrile.

### Example 18

### Synthesis of CPO-11-Y—Pd—Cl

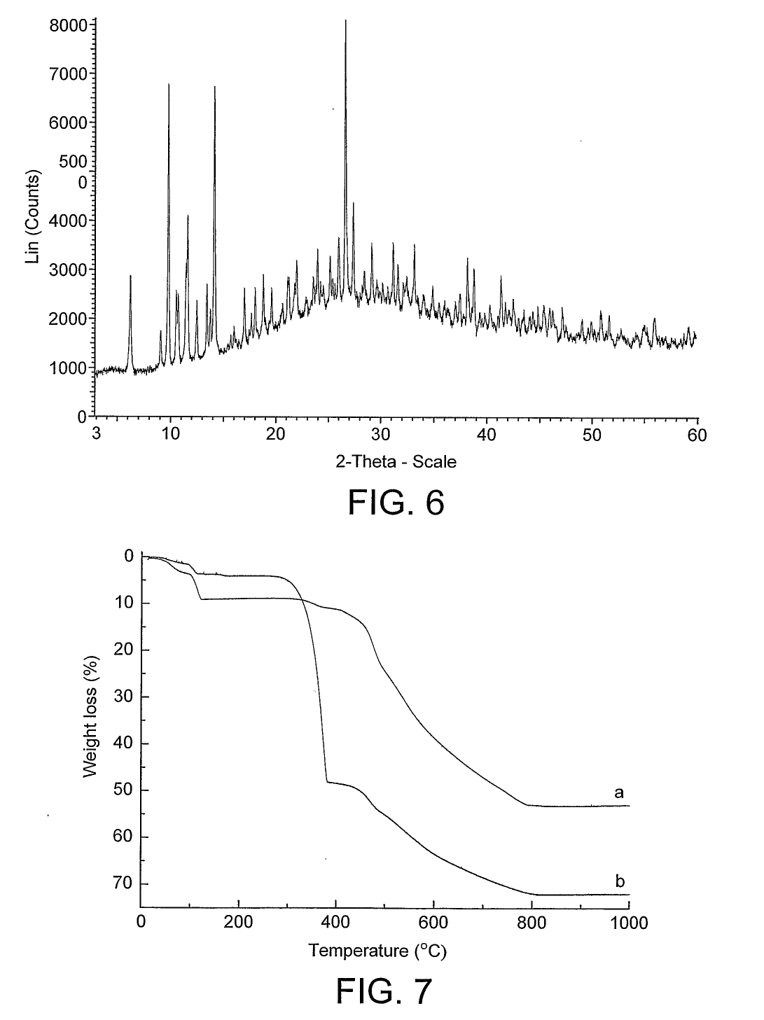

0.04 g K₂PdCl₄, 0.03 g BPDC, 0.17 g Y(NO₃)₃.6H₂O and 9.78 g distilled water were mixed in a Teflon-lined vessel. The vessel was closed in a sealed autoclave and heated to 110° C. for 16 hours. The product was isolated after quenching, filtration, washing with water and drying in air at ambient temperature. The powder X-ray pattern for this is shown in FIG. 6.

### Example 19

### Synthesis of CPO-11-Gd—Pd—Cl

0.04 g K₂PdCl₄, 0.03 g BPDC, 0.20 g Gd(NO₃)₃.6H₂O and 9.76 g distilled water were mixed in a Teflon-lined vessel. The vessel was closed in a sealed autoclave and heated to 110° C. for 17 hours. The product was isolated after quenching, filtration, washing with water and drying in air at ambient temperature.

### Example 20

### Synthesis of CPO-13-La—Pt—Cl

0.10 g K₂PtCl₄, 0.06 g BPDC, 0.06 g LaCl₃.7H₂O and 8.77 g distilled water were mixed in a Teflon-lined vessel. The vessel was closed in a sealed autoclave and placed in a pre-heated oven at 100° C. for 17 hours. The autoclave was quenched after 17 hours. The product was isolated after quenching, filtration, washing with water and drying in air at room temperature.

### Example 21

### Improved Synthesis Method

The product yield of the CPO-11-Gd—Pt—Cl and CPO-11-Y—Pt—Cl frameworks can be improved by using rapid gel heating.

FIG. 7 shows a comparison of the decomposition of CPO-11-Gd—Pt—Cl under N₂ atmosphere of a sample synthesized by using rapid heating (pattern a) and slow heating (pattern b) of the gel. Pattern (b) has a major weight loss around 280° C. which corresponds to decomposition of unconverted ligand.

### Example 22

### Synthesis of CPO-17-Zn—Pt—Cl

41.1 mg K₂PtCl₄, 33.7 mg pyridine-3,5-dicarboxylic acid, 119.7 mg Zn(NO₃)₂.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 23

### Synthesis of CPO-17-Cd—Pt—Cl

41.5 mg K₂PtCl₄, 33.7 mg pyridine-3,5-dicarboxylic acid, 124.4 mg Cd(NO₃)₂.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 24

### Synthesis of CPO-17-Ni—Pt—Cl

41.5 mg K₂PtCl₄, 33.7 mg pyridine-3,5-dicarboxylic acid, 116.4 mg Ni(NO₃)₂.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 25

### Synthesis of CPO-17-Co—Pt—Cl

41.5 mg K₂PtCl₄, 33.7 mg pyridine-3,5-dicarboxylic acid, 118.3 mg Co(NO₃)₂.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 26

### Synthesis of CPO-17-Mn—Pt—Cl

41.5 mg K₂PtCl₄, 33.7 mg pyridine-3,5-dicarboxylic acid, 115.0 mg Mn(NO₃)₂.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 27

### Synthesis of CPO-17-Ba—Pt—Cl

41.5 mg K₂PtCl₄, 33.7 mg pyridine-3,5-dicarboxylic acid, 104.6 mg Ba(NO₃)₂ and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 28

### Synthesis of CPO-18-La—Pt—Cl

41.7 mg K₂PtCl₄, 33.6 mg pyridine-3,5-dicarboxylic acid, 174.5 mg La(NO₃)₃.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 29

### Synthesis of CPO-18-Ce—Pt—Cl

41.7 mg K₂PtCl₄, 33.6 mg pyridine-3,5-dicarboxylic acid, 175.1 mg Ce(NO₃)₃.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 30

### Synthesis of CPO-18-Nd—Pt—Cl

41.7 mg K₂PtCl₄, 33.6 mg pyridine-3,5-dicarboxylic acid, 175.1 mg Nd(NO₃)₃.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 31

### Synthesis of CPO-18-Ce—Pd—Cl

32.5 mg K₂PdCl₄, 33.3 mg pyridine-3,5-dicarboxylic acid, 174.0 mg Ce(NO₃)₃.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 32

### Synthesis of CPO-19-Zn—Pt—Cl

41.7 mg K₂PtCl₄, 33.6 mg pyridine-3,5-dicarboxylic acid, 119.7 mg Zn(NO₃)₃.6H₂O and 10 mL DMF were added to a Teflon-lined vessel. The solution was stirred. The vessel was sealed in a steel autoclave and placed in a pre-heated oven at 100° C. The heating of the oven was stopped after 12 hours. The product was collected after cooling in air to room temperature.

### Example 33

### Synthesis of CPO-19-Cd—Pt—Cl

41.7 mg K₂PtCl₄, 33.6 mg pyridine-3,5-dicarboxylic acid, 123.3 mg Cd(NO₃)₃.4H₂O and 10 mL DMF were added to a Teflon-lined vessel. The solution was stirred. The vessel was sealed in a steel autoclave and placed in a pre-heated oven at 100° C. The heating of the oven was stopped after 12 hours. The product was collected after cooling in air to room temperature.

### Example 34

### Synthesis of CPO-19-Co—Pt—Cl

41.7 mg K₂PtCl₄, 33.6 mg pyridine-3,5-dicarboxylic acid, 115.6 mg Co(NO₃)₃.6H₂O and 10 mL DMF were added to a Teflon-lined vessel. The solution was stirred. The vessel was sealed in a steel autoclave and placed in a pre-heated oven at 100° C. The heating of the oven was stopped after 12 hours. The product was collected after cooling in air to room temperature.

### Example 35

### Synthesis of CPO-19-Mn—Pt—Cl

41.7 mg K₂PtCl₄, 33.6 mg pyridine-3,5-dicarboxylic acid, 115.0 mg Mn(NO₃)₃.6H₂O and 10 mL DMF were added to a Teflon-lined vessel. The solution was stirred. The vessel was sealed in a steel autoclave and placed in a pre-heated oven at 100° C. The heating of the oven was stopped after 12 hours. The product was collected after cooling in air to room temperature.

### Example 36

### Synthesis of CPO-19-Zn—Pd—Cl

32.5 mg K₂PdCl₄, 33.3 mg pyridine-3,5-dicarboxylic acid, 119.7 mg Zn(NO₃)₃.6H₂O and 10 mL DMF were added to a Teflon-lined vessel. The solution was stirred. The vessel was sealed in a steel autoclave and placed in a pre-heated oven at 100° C. The heating of the oven was stopped after 12 hours. The product was collected after cooling in air to room temperature.

### Example 37

### Synthesis of CPO-20-Nd—Pt—Cl

41.7 mg K₂PtCl₄, 33.6 mg pyridine-3,5-dicarboxylic acid, 175.1 mg Nd(NO₃)₃.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 38

### Synthesis of CPO-21-Nd—Pt

41.7 mg K₂PtCl₄, 33.6 mg pyridine-2,4-dicarboxylic acid, 175.1 mg Nd(NO₃)₃.6H₂O and 10 mL distilled water were added to a Teflon-lined vessel. The solution was stirred. The vessel was sealed in a steel autoclave and placed in a pre-heated oven at 120° C. The heating of the oven was stopped after 12 hours. After cooling in air to room temperature, the product was washed with distilled water and dried in air.

### Example 39

### Catalytic Testing: Hydrogenation of Ethene

All catalytic tests were performed under similar conditions. Tests were done in a quartz batch reactor. The samples, containing 60.7 μmol platinum compound, were placed on a bed of quartz wool, and small amount of quartz wool was placed above the sample. The reactor volumes above and below the catalyst bed were reduced by placing two 0.3 mm thick quartz tubes in the reactor. The reactor was first flushed with helium gas (15 ml/min) for 20 minutes and then with 1:1 (vol) mixture of C₂H₄ and H₂ (20 ml/min) for 10 minutes. The reactor was then sealed under the pressure of 1.5 bar of C₂H₄/H₂ gas mixture. After heating the reactor to 100° C. (5° C./min), gas samples of about 0.1 ml were taken out from the reactor and analysed with GC-MS.

The results are shown in FIG. 8. The catalysts used were those of Examples 29 (▪), 25 (), 4 (▾), and 33 [symbol] . A blind run was also carried out with no catalyst (o).

1. A porous heterogeneous catalyst material comprising a framework of inorganic cornerstones connected by organic bridges, wherein said organic bridges comprise compounds having a complexed catalytically active metal.

2. The catalyst material as claimed in claim 1 wherein said framework is three-dimensional.

3. The catalyst material as claimed in claim 1 wherein the complexed catalytically active metal is selected from the group consisting of group 4, group 6 and group 8 metals.

4. The catalyst material as claimed in claim 3 wherein the complexed catalytically active metal is selected from the group consisting of palladium and platinum.

5. The catalyst material as claimed in claim 1 wherein the inorganic cornerstones contain at least one group 2, transition, lanthanide or actinide metal atom.

6. The catalyst material as claimed in claim 5 wherein the inorganic cornerstones contain at least one metal atom selected from the group consisting of Hf, Zr, Ti, Y, Sc, La, Gd, Sm, Dy, Ho, Er, Cu, Zn, Cd, Ni, Co, Fe, Mn, Cr, Ce, Ba, Pb and Nd.

7. A process for preparing a catalyst material according to claim 1, said process comprising
complexing an inorganic cornerstone with a multivalent organometallic ligand in a liquid solvent,
optionally transmetallating the product to introduce a catalytically active metal, and
removing the solvent.

8. (canceled)

9. A process for the catalysed transformation of a hydrocarbon, comprising contacting the hydrocarbon with as a catalyst comprising the catalyst material according to claim 1.
